(19) United States
(12) Patent Application Publication
(10) Pub. No.: US 2009/0312713 A1
(43) Pub. Date: Dec. 17, 2009

(54) METHOD FOR MARKING PHARMACEUTICAL ARTICLES

(75) Inventors: Fabrice Greutert, Bussy-Chardonney (CH); Bernard Anthoine, Reignier (FR)

Correspondence Address: Nixon Peabody LLP, P.O. Box 60610, Palo Alto, CA, 94306, US

(73) Assignee: ARES TRADING SA, Aubonne (CH)

(21) Appl. No.: 12/297,633

(22) PCT Filed: Apr. 19, 2007
(86) PCT No.: PCT/IB2007/001016

§ 371 Date: Feb. 20, 2009

Related U.S. Application Data
(60) Provisional application No. 60/794,565, filed on Apr. 24, 2006.

(30) Foreign Application Priority Data
Apr. 20, 2006 (EP) 06008144.5

(51) Int. Cl. A61M 5/31; B65D 83/04; A61M 3/00; B44F 1/10

(52) U.S. Cl. 604/189; 206/534; 427/2.1; 427/2.28; 428/29

(57) ABSTRACT

A method for marking pharmaceutical articles is characterized by comprising marking the pharmaceutical articles with an ink that is invisible under normal light conditions and that is visible under specific light conditions, such as under UV light.

### CROSS REFERENCE TO RELATED APPLICATIONS OR PRIORITY CLAIM

This application is a national phase of International Application No. PCT/IB2007/001016, entitled “METHOD FOR MARKING PHARMACEUTICAL ARTICLES”, which was filed on Apr. 19, 2007, which claims priority of European Patent Application No. 06008144.5, filed Apr. 20, 2006 and U.S. Provisional Application No. 60/794,565, filed Apr. 24, 2006.

### FIELD OF INVENTION

The present invention pertains to a method for marking pharmaceutical articles.

### BACKGROUND OF INVENTION

Marking pharmaceutical articles is generally required, particularly for permitting traceability of the said articles and for detecting product imitation.

One purpose of the present invention is to provide a method that can make both hidden and easily readable markings on pharmaceutical articles.

1. To this end, there is provided a method for marking pharmaceutical articles, characterized by comprising marking the pharmaceutical articles with an ink that is invisible under normal light conditions and that is visible under specific light conditions.

2. In an embodiment, the present invention resides in a method as defined at point 1 above wherein said ink is visible under ultra-violet light.

3. In another embodiment, the present invention resides in a method as defined at point 1 or 2 above wherein the pharmaceutical articles are medication containers.

4. In another embodiment, the present invention resides in a method as defined at point 3 above wherein the medication containers are prefilled with medication

5. In another embodiment, the present invention resides in a method as defined at point 4 above wherein the step of marking the pharmaceutical articles with said ink is performed in a production line, downstream of a portion of the production line in which the medication containers are filled with said medication.

6. In another embodiment, the present invention resides in a method as defined at any one of points 3 to 5 above wherein the pharmaceutical articles are syringes.

7. In another embodiment, the present invention resides in a method as defined at point 6 above wherein the step of marking the pharmaceutical articles with said ink comprises marking a rigid plastic needle shield of said syringes.

8. In another embodiment, the present invention resides in a method as defined at any one of points 3 to 7 above wherein the medication containers are transparent.

9. In another embodiment, the present invention resides in a method as defined at any one of points 3 to 8 above wherein said medication is a protein of therapeutic interest.

10. In another embodiment, the present invention resides in a method as defined at point 9 above wherein said protein of therapeutic interest is selected from the group consisting of chorionic gonadotropin, follicle-stimulating hormone, lutropin-choriogonadotropic hormone, thyroid stimulating hormone, human growth hormone, interferons (e.g., interferon beta-1a or interferon beta-1b, or peginterferon alfa-2a), interferon receptors (e.g., interferon gamma receptor), TNF receptors p55 and p75, interleukins (e.g., interleukin-2 or interleukin-11), interleukin binding proteins (e.g., interleukin-18 binding protein), anti-CD11a antibodies, erythropoietin, granulocyte colony stimulating factor (e.g., filgrastim or pegfilgrastim), granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, insulin lispro, glargine insulin, insulin Detemir, Factor VIII, somatropin, bone morphogenetic protein-2, platelet-derived growth factor, hirudin, epoietin, darbepoetin alfa, recombinant LFA-3/IgG1 fusion protein, glucocerebrosidase, agalsidase beta, etanercept, imiglucerase, drotrecogin alpha, alefacept, pegfilgrastim, beclapermin, trafermin, ancetism, a monoclonal antibody (e.g. trastuzumab, or omalizumab, or efalizumab, or infliximab, or rituximab, or tositumomab, or ibritumomab tiuxetan, or bevacizumab, or cetuximab, or natalizumab, or adalimumab) and muteins, fragments, soluble forms, functional derivatives, fusion proteins thereof.

11. In another embodiment, the present invention resides in a method as defined at point 10 above wherein said protein of therapeutic interest is chorionic gonadotropin or follicle-stimulating hormone or lutropin-choriogonadotropic hormone, or human growth hormone, or interferon beta-1b, or efalizumab, or cetuximab.

12. In another embodiment, the present invention resides in a method as defined at any one of points 1 to 11 above wherein the step of marking the pharmaceutical articles with said ink comprises printing coded information on said pharmaceutical articles.

13. In another embodiment, the present invention resides in a method as defined at point 12 above wherein said coded information comprises one or more parallel lines extending substantially over an entire circumference of the pharmaceutical articles.

14. In another embodiment, the present invention resides in a method as defined at point 13 above wherein said one or more parallel lines are indicative of a type of medication included in the pharmaceutical articles.

15. In another embodiment, the present invention resides in a method as defined at any one of points 12 to 14 above wherein said coded information comprises one or more alpha-numeric characters indicative of a batch of the pharmaceutical articles.

16. In another embodiment, the present invention resides in a method as defined at any one of points 1 to 15 above wherein the step of marking the pharmaceutical articles with said ink is performed by means of several printer heads projecting said ink onto different sides of said pharmaceutical articles.

17. The present invention also provides a pharmaceutical article marked by a method as defined at any one of points 1 to 16 above.

18. The present invention also provides a medication container marked by a method as defined at any one of points 1 to 16 above.

19. The present invention also provides an injection device comprising a medication container as defined at point 18 above and means for reading the marking provided on said medication container.

20. In an embodiment, the present invention resides in an injection device as defined at point 19 above wherein said reading means are optical means

21. In another embodiment, the present invention resides in an injection device as defined at point 19 or 20 above wherein the marking provided on said medication container comprises one or more parallel lines extending over substantially the entire circumference of said medication container so as to be readable by said reading means irrespective of the angular position of said medication container in said injection device.

### BRIEF DESCRIPTION OF THE DRAWINGS

Other features and advantages of the present invention will appear upon reading the following detailed description made with reference to the appended diagrammatic drawings in which:

In the drawings:

FIG. 1 is a top view of a device for marking pharmaceutical articles according to the method of the present invention,

FIG. 2 is a front view of the device shown in FIG. 1,

FIG. 3 is a front view of four plastic rigid needle shields marked with the method of the present invention,

FIG. 4 is a front view of a medication cartridge marked with the method of the present invention,

FIG. 5 is a diagrammatic view showing an injection device incorporating the cartridge shown in FIG. 4.

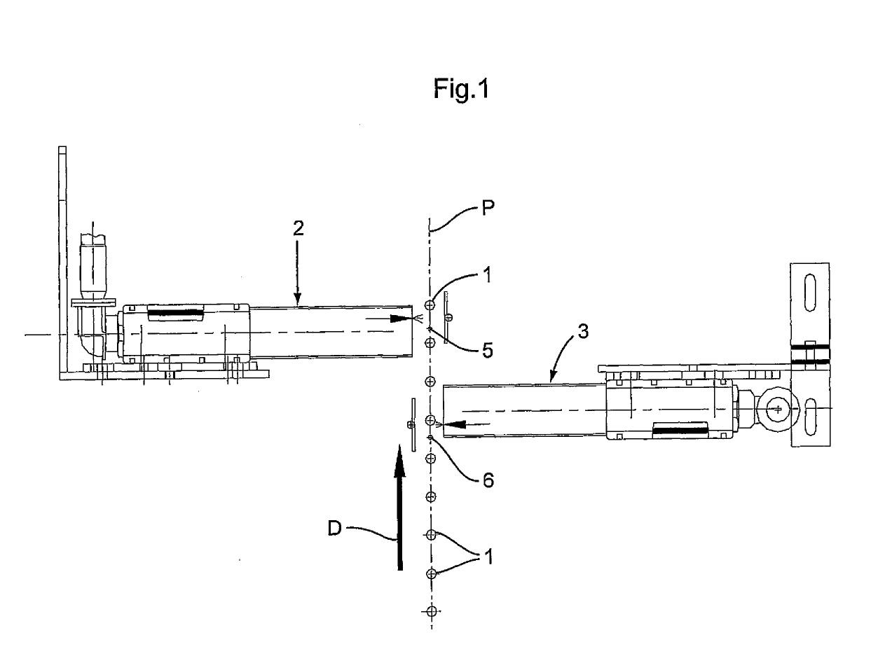

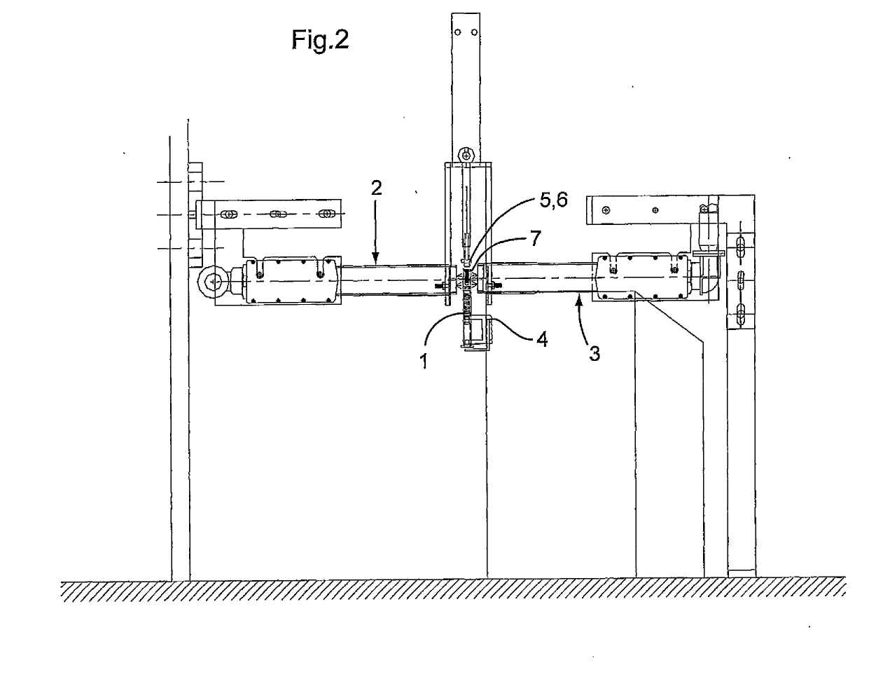

Referring to FIGS. 1 and 2, a device for marking pharmaceutical articles, i.e. prefilled syringes 1 in the example shown, comprises a printer having two printer heads 2, 3. The printer heads 2, 3 are placed in a production line, downstream of a portion of the production line where the syringes 1 are filled with medication and closed with a stopper. The syringes 1 are held by grippers 4 which are moved along a path P in the direction designated by D by a drive mechanism including a motor (not shown). The printer heads 2, 3 are located on either side of the syringes' path P and are offset relative to one another in the direction D so as not to project ink towards one another. The printer controls the printer heads 2, 3 so that a marking is printed on the syringes 1 as these latter pass in front of the printer heads 2, 3. Information on the position and displacement rate of the syringes 1 is provided to the printer by two sensors 5, 6, such as optical fibre sensors, placed just upstream of the nozzles of the printer heads 2, 3 respectively and above the syringes' path P, and by an encoder (not shown). The sensors 5, 6 detect the passage of syringes 1 below them. The encoder determines the displacement rate of the syringes 1 based on the rate of the driving motor. Downstream of the marking device 2 to 6 in the production line, the syringes 1 are put into blisters (packaging).

The marking is printed on a surface of the syringes 1 on which the ink can sufficiently adhere. A suitable surface for this purpose is the external surface of the plastic rigid needle shield, designated by 7. Glass surfaces, i.e. typically the body surface of syringes 1, are not suitable because they are generally covered with a silicone film.

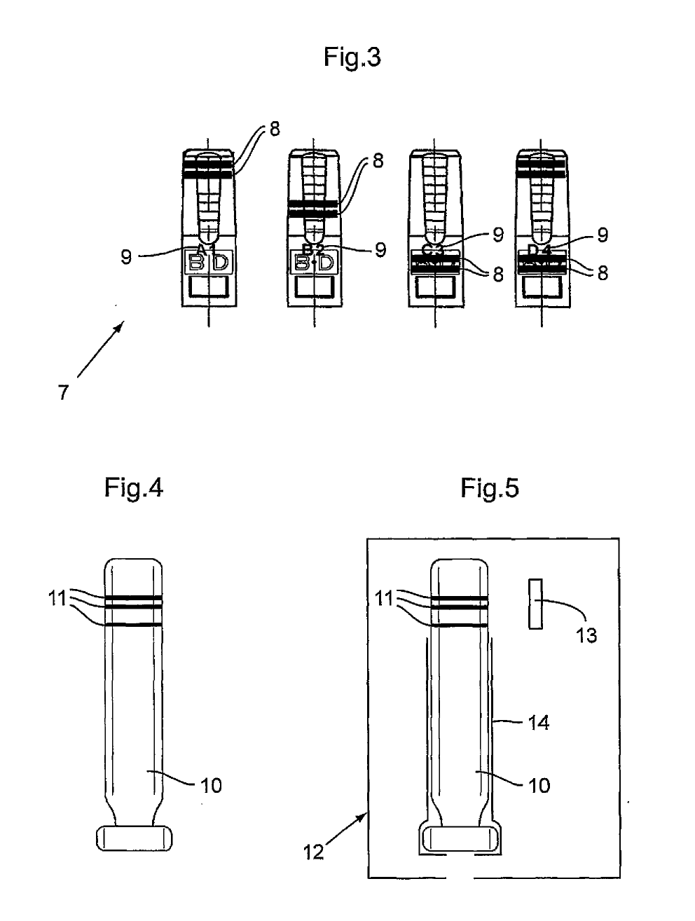

Referring to FIG. 3, the marking printed on the plastic rigid needle shield 7 of syringes 1 consists of coded information in the form of one or more parallel lines 8 extending in the circumferential direction of the shield 7 and one or more alpha-numeric characters 9. The position and number of the parallel lines 8 on a given needle shield 7 may be indicative of the type of medication contained in the corresponding syringe 1, whereas the alpha-numeric characters 9 may be indicative of the batch and the production site of the medication contained in the said syringe. Thanks to the two printer heads 2, 3, which are arranged to project ink onto two opposite sides of the needle shields 7, the parallel lines 8 can extend over substantially the entire circumference of the needle shields 7 and can thus form circles surrounding the needle shields 7. In this manner, when the syringes 1 are in their blisters, the parallel lines 8 can be seen through the transparent undersurface of the blisters irrespective of the angular position of the syringes 1. The alpha-numeric characters 9 are also printed on the needle shields 7 by both of the printer heads 2, 3 so that they can be seen on two opposite sides of the needle shields 7.

The ink used in the present invention for marking the syringes 1 is an invisible ink, i.e. an ink that is invisible under normal (white) light conditions but that is visible under specific light conditions such as under ultra-violet (UV) light. An example of a suitable invisible ink is the ink commercialized by the company IMAJE under reference 5535. Such an ink emits blue luminescent light when excited by UV light.

Thus, the marking according to the present invention does not affect the visible appearance of the syringes 1. The marking consists of hidden information which may be used for traceability, e.g. to identify a determined batch of syringes 1 after a problem has been found out in the production process, or in the fight against infringement, to distinguish the syringes 1 and the medication contained therein from infringing ones. As it is invisible, the marking may take up a large area on the surface of the needle shields 7, to be easily readable when viewed under a UV lamp. The marking may even be superposed to visible information, as shown in FIG. 3, to gain room on the needle shield surface.

The marking according to the invention is also particularly advantageous when applied on transparent syringes. Transparent syringes enable visually controlling the medication to detect any turbidity. As it is invisible, the marking according to the invention does not impede such a control.

The marking according to the invention may be read by human eyes by illuminating the syringes 1, particularly the needle shields 7, with UV light. Alternatively, the marking may be read by an optical scanner (not shown) which emits UV light to excite the invisible ink and detects the visible, luminescent light emitted in response by the ink. In this latter case, the lines 8 form a bar code readable by the optical scanner.

Typically, the medication contained by the syringes 1 includes a protein of therapeutic interest. The protein of therapeutic interest may be, for example, a naturally secreted protein, a normally cytoplasmic protein, a normally transmembrane protein, or a human or a humanized antibody. When the protein of interest is a normally cytoplasmic or a normally transmembrane protein, the protein has preferably been engineered in order to become soluble. The polypeptide of interest may be of any origin. Preferred polypeptides of interest are of human origin.

Preferably, the protein of therapeutic interest is selected from the group consisting of chorionic gonadotropin, follicle-stimulating hormone, lutropin-choriogonadotropic hormone, thyroid stimulating hormone, human growth hormone, interferons (e.g., interferon beta-1a or interferon beta-1b, or peginterferon alfa-2a), interferon receptors (e.g., interferon gamma receptor), TNF receptors p55 and p75, interleukins (e.g., interleukin-2 or interleukin-11), interleukin binding proteins (e.g., interleukin-18 binding protein), anti-CD11a antibodies, erythropoietin, granulocyte colony stimulating factor (e.g., filgrastim or pegfilgrastim), granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, insulin lispro, glargine insulin, insulin Detemir, Factor VIII, somatropin, bone morphogenetic protein-2, platelet-derived growth factor, hirudin, epoietin, darbepoetin alfa, recombinant LFA-3/IgG1 fusion protein, glucocerebrosidase, agalsidase beta, etanercept, imiglucerase, drotrecogin alpha, alefacept, pegfilgrastim, beclapermin, trafermin, ancetism, a monoclonal antibody (e.g. trastuzumab, or omalizumab, or efalizumab, or infliximab, or rituximab, or tositumomab, or ibritumomab tiuxetan, or bevacizumab, or cetuximab, or natalizumab, or adalimumab) and muteins, fragments, soluble forms, functional derivatives, fusion proteins thereof.

The marking method according to the present invention is not limited to syringes. It is indeed clear that this method may be applied to other kinds of pharmaceutical articles, such as other medication containers (cartridges, ampoules, vials, etc.) as well as medication tablets and capsules. FIG. 4 shows, by way of example, a cartridge 10 having a marking 11 made with an invisible ink. If the external surface of the cartridge 10 is of glass rather than plastic, the marking 11 is printed on an adhesive label provided on the said surface. In a preferred embodiment, the cartridge 10 is intended to be used in an injection device as described in WO 2005/077441 and diagrammatically shown in FIG. 5 at reference 12, and the marking 11 consists of a bar code readable by a small optical scanner 13 provided in that injection device 12. Detection of the marking 11 by the optical scanner 13 may be indicative of proper insertion of the cartridge 10 in the injection device 12 and/or of the type, amount, manufacturer, batch and/or expiration date of the medication contained in the cartridge 10. The marking 11 is preferably in the form of one or more parallel lines that extend over substantially the entire circumference of the cartridge 10 so as to be readable by the optical scanner 13 irrespective of the angular position of the cartridge 10 in its holder, designated by 14.

1. A method for marking pharmaceutical articles, characterized by comprising marking the pharmaceutical articles with an ink that is invisible under normal light conditions and that is visible under specific light conditions.

2. The method according to claim 1, characterized in that said ink is visible under ultra-violet light.

3. The method according to claim 1, characterized in that the pharmaceutical articles are medication containers (1; 10).

4. The method according to claim 3, characterized in that the medication containers (1; 10) are prefilled with medication.

5. The method according to claim 4, characterized in that the step of marking the pharmaceutical articles with said ink is performed in a production line, downstream of a portion of the production line in which the medication containers (1) are filled with said medication.

6. The method according to claim 3, characterized in that the pharmaceutical articles are syringes (1).

7. The method according to claim 6, characterized in that the step of marking the pharmaceutical articles with said ink comprises marking a rigid plastic needle shield (7) of said syringes (1).

8. The method according to claim 3, characterized in that said medication containers (1; 10) are transparent.

9. The method according to claim 3, characterized in that said medication is a protein of therapeutic interest.

10. The method according to claim 9, characterized in that said protein of therapeutic interest is selected from the group consisting of chorionic gonadotropin, follicle-stimulating hormone, lutropin-choriogonadotropic hormone, thyroid stimulating hormone, human growth hormone, interferons (e.g., interferon beta-1a or interferon beta-1b, or peginterferon alfa-2a), interferon receptors (e.g., interferon gamma receptor), TNF receptors p55 and p75, interleukins (e.g., interleukin-2 or interleukin-11), interleukin binding proteins (e.g., interleukin-18 binding protein), anti-CD11a antibodies, erytbropoietin, granulocyte colony stimulating factor (e.g., filgrastim or pegfilgrastim), granulocyte-macrophage colony-stimulating factor, pituitary peptide hormones, menopausal gonadotropin, insulin-like growth factors (e.g., somatomedin-C), keratinocyte growth factor, glial cell line-derived neurotrophic factor, thrombomodulin, basic fibroblast growth factor, insulin, insulin lispro, glargine insulin, insulin Detemir, Factor VIII, somatropin, bone morphogenetic protein-2, platelet-derived growth factor, hirudin, epoietin, darbepoetin alfa, recombinant LFA-3/IgG1 fusion protein, glucocerebrosidase, agalsidase beta, etanercept, imiglucerase, drotrecogin alpha, alefacept, pegfilgrastim, beclapermin, trafermin, ancetism, a monoclonal antibody (e.g. trastuzumab, or omalizumab, or efalizumab, or infliximab, or rituximab, or tositumomab, or ibritumomab tiuxetan, or bevacizumab, or cetuximab, or natalizumab, or adalimumab) and muteins, fragments, soluble forms, functional derivatives, fusion proteins thereof.

11. The method according to claim 10, characterized in that said protein of therapeutic interest is chorionic gonadotropin or follicle-stimulating hormone or lutropin-choriogonadotropic hormone, or human growth hormone, or interferon beta-1b, or efalizumab, or cetuximab.

12. The method according to claim 1, characterized in that the step of marking the pharmaceutical articles with said ink comprises printing coded information (8, 9; 11) on said pharmaceutical articles.

13. The method according to claim 12, characterized in that said coded information comprises one or more parallel lines (8; 11) extending substantially over an entire circumference of the pharmaceutical articles.

14. The method according to claim 13, characterized in that said one or more parallel lines (8) are indicative of a type of medication included in the pharmaceutical articles.

15. The method according to claim 12, characterized in that said coded information comprises one or more alpha-numeric characters (9) indicative of a batch of the pharmaceutical articles.

16. The method according to claim 1, characterized in that the step of marking the pharmaceutical articles with said ink is performed by means of several printer heads (2, 3) projecting said ink onto different sides of said pharmaceutical articles (1).

17. A pharmaceutical article marked by the method according to claim 1.

18. A medication container marked by the method according to claim 1.

19. An injection device comprising a medication container according to claim 18 and including means for reading the marking provided on said medication container.

20. The injection device according to claim 19, characterized in that said reading means are optical means.

21. The injection device according to claim 19, characterized in that the marking provided on said medication container comprises one or more parallel lines extending over substantially the entire circumference of said medication container so as to be readable by said reading means irrespective of the angular position of said medication container in said injection device.
